SEQUENCE LISTING (56 sequences)

SEQ ID NO: 1 - DNA, 373 bp, Artificial Sequence
  DD
acggccgccagtgtgctggccaccatgggagtgcaggtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaaccggaagccggcgcaatgggcagcagcctgg

SEQ ID NO: 2 - DNA, 48 bp, Artificial Sequence
  DDT2-F
ggagcttctaaaaccggaagccggcatggaggaagtatgtgattcatc

SEQ ID NO: 3 - DNA, 42 bp, Artificial Sequence
  DDT2-R
gccagcacactggcggccgttccgcagcttttagagcagaag

SEQ ID NO: 4 - DNA, 57 bp, Artificial Sequence
  V-F
cacctctgctctctcttcattttattatatattagtcacgatatctataacaagaaa

SEQ ID NO: 5 - DNA, 59 bp, Artificial Sequence
  V-R
ctctgggatccagaatgtttaaaagttttgttactttatagaagaaattttgagttttt

SEQ ID NO: 6 - DNA, 36 bp, Artificial Sequence
  1-F
tttaaacattctggatcccagaggtgtaaagtactt

SEQ ID NO: 7 - DNA, 44 bp, Artificial Sequence
  1-R
cacattccacagtctaatacgactcactatagggagaccaaggg

SEQ ID NO: 8 - DNA, 35 bp, Artificial Sequence
  2-F
gtcgtattagactgtggaatgtgtgtcagttaggg

SEQ ID NO: 9 - DNA, 20 bp, Artificial Sequence
  2-R
tggcggcaggcggccgacct

SEQ ID NO: 10 - DNA, 20 bp, Artificial Sequence
  3-F
ggccgcctgccgccaccatc

SEQ ID NO: 11 - DNA, 26 bp, Artificial Sequence
  3-R
attatgatcctcgtcaggcaccgggc

SEQ ID NO: 12 - DNA, 41 bp, Artificial Sequence
  4-F
cggtgcctgacgaggatcataatcagccataccacatttgt

SEQ ID NO: 13 - DNA, 48 bp, Artificial Sequence
  4-R
atacgaagttataatttcctttttgttaagtgacctaattaacaggag

SEQ ID NO: 14 - DNA, 108 bp, Artificial Sequence
  F5
ataacttcgtatagcatacattatacgaagttatggcgcgccattctagaggtacctaaccggtatccatggagataacttcgtatagcatacattatacgaagttat

SEQ ID NO: 15 - DNA, 39 bp, Artificial Sequence
  6-F
tacgaagttatatcaattaaccctcactaaagggagacc

SEQ ID NO: 16 - DNA, 57 bp, Artificial Sequence; identical to SEQ ID NO: 4
  6-R

SEQ ID NO: 17 - DNA, 38 bp, Artificial Sequence
  UCOE-F
aagttatgtaacgggtacctggccctccgcgcctacag

SEQ ID NO: 18 - DNA, 38 bp, Artificial Sequence
  UCOE-R
cgaagttatggcgcgccattggagacgccgtggccccc

SEQ ID NO: 19 - DNA, 43 bp, Artificial Sequence
  pT3-F
aacaacaattgctaattaagatctcgagggaatgaaagacccc

SEQ ID NO: 20 - DNA, 40 bp, Artificial Sequence
  pT3-R
aatcaatgtcaacaactagtatcgatatgcatgctttgca

SEQ ID NO: 21 - DNA, 58 bp, Artificial Sequence
  F1-F
tcgatactagttgttgacattgattattgactagttattaatagtaatcaattacggg

SEQ ID NO: 22 - DNA, 38 bp, Artificial Sequence
  F1-R
agcttgaattcgaagctctgcttatatagacctcccac

SEQ ID NO: 23 - DNA, 39 bp, Artificial Sequence
  F2-F
ataagcagagcttcgaattcaagctgctagcaaggatcc

SEQ ID NO: 24 - DNA, 44 bp, Artificial Sequence
  F2-R
gagatcttaattagcaattgttgttgttaacttgtttattgcag

SEQ ID NO: 25 - DNA, 53 bp, Artificial Sequence
  SPLNK-HP
ggaattctcgggtgccaaggaactccagtcacttttttttttcaaaaaaaaaa

SEQ ID NO: 26 - DNA, 55 bp, Artificial Sequence
  SPLNK-LE
  misc_feature (54)..(55): phosphorothioesther bond
gttcccatggtactactcatagtgactggagttccttggcacccgagaattccat

SEQ ID NO: 27 - DNA, 30 bp, Artificial Sequence
  5-1-F
  misc_feature (29)..(30): phosphorothioesther bond
cgctatttagaaagagagagcaatatttca

SEQ ID NO: 28 - DNA, 21 bp, Artificial Sequence
  SPL-1-R
  misc_feature (20)..(21): phosphorothioesther bond
gttcccatggtactactcata

SEQ ID NO: 29 - DNA, 95 bp, Artificial Sequence
  PB5-2-P501
  misc_feature (94)..(95): phosphorothioesther bond
aatgatacggcgaccaccgagatctacatatagcctacactctttccctacacgacgctcttccgatctcatgcgtcaattttacgcagactatc

SEQ ID NO: 30 - DNA, 95 bp, Artificial Sequence
  PB5-2-P502
  misc_feature (94)..(95): phosphorothioesther bond
aatgatacggcgaccaccgagatctacaatagaggcacactctttccctacacgacgctcttccgatctcatgcgtcaattttacgcagactatc

SEQ ID NO: 31 - DNA, 95 bp, Artificial Sequence
  PB5-2-P503
  misc_feature (94)..(95): phosphorothioesther bond
aatgatacggcgaccaccgagatctacacctatcctacactctttccctacacgacgctcttccgatctcatgcgtcaattttacgcagactatc

SEQ ID NO: 32 - DNA, 95 bp, Artificial Sequence
  PB5-2-P504
  misc_feature (94)..(95): phosphorothioesther bond
aatgatacggcgaccaccgagatctacaggctctgaacactctttccctacacgacgctcttccgatctcatgcgtcaattttacgcagactatc

SEQ ID NO: 33 - DNA, 95 bp, Artificial Sequence
  PB5-2-P505
  misc_feature (94)..(95): phosphorothioesther bond
aatgatacggcgaccaccgagatctacaaggcgaagacactctttccctacacgacgctcttccgatctcatgcgtcaattttacgcagactatc

SEQ ID NO: 34 - DNA, 95 bp, Artificial Sequence
  PB5-2-P506
  misc_feature (94)..(95): phosphorothioesther bond
aatgatacggcgaccaccgagatctacataatcttaacactctttccctacacgacgctcttccgatctcatgcgtcaattttacgcagactatc

SEQ ID NO: 35 - DNA, 95 bp, Artificial Sequence
  PB5-2-P507
  misc_feature (94)..(95): phosphorothioesther bond
aatgatacggcgaccaccgagatctacacaggacgtacactctttccctacacgacgctcttccgatctcatgcgtcaattttacgcagactatc

SEQ ID NO: 36 - DNA, 95 bp, Artificial Sequence
  PB5-2-P508
  misc_feature (94)..(95): phosphorothioesther bond
aatgatacggcgaccaccgagatctacagtactgacacactctttccctacacgacgctcttccgatctcatgcgtcaattttacgcagactatc

SEQ ID NO: 37 - DNA, 95 bp, Artificial Sequence
  T25-2-P501
  misc_feature (94)..(95): phosphorothioesther bond
aatgatacggcgaccaccgagatctacatatagcctacactctttccctacacgacgctcttccgatctatttttgagtactttttacacctctg

SEQ ID NO: 38 - DNA, 95 bp, Artificial Sequence
  T25-2-P502
  misc_feature (94)..(95): phosphorothioesther bond
aatgatacggcgaccaccgagatctacaatagaggcacactctttccctacacgacgctcttccgatctatttttgagtactttttacacctctg

SEQ ID NO: 39 - DNA, 95 bp, Artificial Sequence
  T25-2-P503
  misc_feature (94)..(95): phosphorothioesther bond
aatgatacggcgaccaccgagatctacacctatcctacactctttccctacacgacgctcttccgatctatttttgagtactttttacacctctg

SEQ ID NO: 40 - DNA, 95 bp, Artificial Sequence
  T25-2-P504
  misc_feature (94)..(95): phosphorothioesther bond
aatgatacggcgaccaccgagatctacaggctctgaacactctttccctacacgacgctcttccgatctatttttgagtactttttacacctctg

SEQ ID NO: 41 - DNA, 95 bp, Artificial Sequence
  T25-2-P505
  misc_feature (94)..(95): phosphorothioesther bond
aatgatacggcgaccaccgagatctacaaggcgaagacactctttccctacacgacgctcttccgatctatttttgagtactttttacacctctg

SEQ ID NO: 42 - DNA, 95 bp, Artificial Sequence
  T25-2-P506
  misc_feature (94)..(95): phosphorothioesther bond
aatgatacggcgaccaccgagatctacataatcttaacactctttccctacacgacgctcttccgatctatttttgagtactttttacacctctg

SEQ ID NO: 43 - DNA, 95 bp, Artificial Sequence
  T25-2-P507
  misc_feature (94)..(95): phosphorothioesther bond
aatgatacggcgaccaccgagatctacacaggacgtacactctttccctacacgacgctcttccgatctatttttgagtactttttacacctctg

SEQ ID NO: 44 - DNA, 95 bp, Artificial Sequence
  T25-2-P508
  misc_feature (94)..(95): phosphorothioesther bond
aatgatacggcgaccaccgagatctacagtactgacacactctttccctacacgacgctcttccgatctatttttgagtactttttacacctctg

SEQ ID NO: 45 - DNA, 51 bp, Artificial Sequence
  SPL-2-P701
  misc_feature (50)..(51): phosphorothioesther bond
caagcagaagacggcatacgagatcgagtaatgtgactggagttccttggc

SEQ ID NO: 46 - DNA, 51 bp, Artificial Sequence
  SPL-2-P702
  misc_feature (50)..(51): phosphorothioesther bond
caagcagaagacggcatacgagattctccggagtgactggagttccttggc

SEQ ID NO: 47 - DNA, 51 bp, Artificial Sequence
  SPL-2-P703
  misc_feature (50)..(51): phosphorothioesther bond
caagcagaagacggcatacgagataatgagcggtgactggagttccttggc

SEQ ID NO: 48 - DNA, 51 bp, Artificial Sequence
  SPL-2-P704
  misc_feature (50)..(51): phosphorothioesther bond
caagcagaagacggcatacgagatggaatctcgtgactggagttccttggc

SEQ ID NO: 49 - DNA, 51 bp, Artificial Sequence
  SPL-2-P705
  misc_feature (50)..(51): phosphorothioesther bond
caagcagaagacggcatacgagatttctgaatgtgactggagttccttggc

SEQ ID NO: 50 - DNA, 51 bp, Artificial Sequence
  SPL-2-P706
  misc_feature (50)..(51): phosphorothioesther bond
caagcagaagacggcatacgagatacgaattcgtgactggagttccttggc

SEQ ID NO: 51 - DNA, 51 bp, Artificial Sequence
  SPL-2-P707
  misc_feature (50)..(51): phosphorothioesther bond
caagcagaagacggcatacgagatagcttcaggtgactggagttccttggc

SEQ ID NO: 52 - DNA, 51 bp, Artificial Sequence
  SPL-2-P708
  misc_feature (50)..(51): phosphorothioesther bond
caagcagaagacggcatacgagatgcgcattagtgactggagttccttggc

SEQ ID NO: 53 - DNA, 51 bp, Artificial Sequence
  SPL-2-P709
  misc_feature (50)..(51): phosphorothioesther bond
caagcagaagacggcatacgagatcatagccggtgactggagttccttggc

SEQ ID NO: 54 - DNA, 51 bp, Artificial Sequence
  SPL-2-P710
  misc_feature (50)..(51): phosphorothioesther bond
caagcagaagacggcatacgagatttcgcggagtgactggagttccttggc

SEQ ID NO: 55 - DNA, 51 bp, Artificial Sequence
  SPL-2-P711
  misc_feature (50)..(51): phosphorothioesther bond
caagcagaagacggcatacgagatgcgcgagagtgactggagttccttggc

SEQ ID NO: 56 - DNA, 51 bp, Artificial Sequence
  SPL-2-P712
  misc_feature (50)..(51): phosphorothioesther bond
caagcagaagacggcatacgagatctatcgctgtgactggagttccttggc

(19) United States
(12) Patent Application Publication
(10) Pub. No.: US 2024/0124872 A1
(43) Pub. Date: Apr. 18, 2024

(54) IDENTIFICATION OF GENOMIC TARGETS

(71) Applicant: University of Leeds, Leeds, Yorkshire (GB)

(72) Inventor: Viktor Lukacs, Leeds, Yorkshire (GB)

(73) Assignee: University of Leeds, Leeds, Yorkshire (GB)

(21) Appl. No.: 18/278,366

(22) PCT Filed: Feb. 22, 2022
(86) PCT No.: PCT/GB2022/050494

§ 371 Date: Aug. 22, 2023

(30) Foreign Application Priority Data
Feb. 23, 2021 (GB) 2102557.2

(51) Int. Cl. C12N 15/10

(52) U.S. Cl. CPC C12N 15/1082; C12N 15/1065; C12N 15/1086

(57) ABSTRACT

The invention relates to a method of identifying a genomic target for a test stimulus that is capable of modulating a cell phenotype, comprising: a) providing a population of cells that are phenotypically negative in response to the test stimulus; b) modifying the population of cells by random insertion into the cell genome of a gain-in-function construct; c) contacting the modified cell population with the test stimulus; d) screening the exposed modified cell population to identify modified cells that are phenotypically positive in response to the test stimulus; and e) identify a gene or genes associated with said positive phenotype thereby identifying the genomic target of the test stimulus.

The present invention provides a method of identifying a genomic target for a test stimulus that is capable of modulating a cell phenotype.

### BACKGROUND

Identifying of new genes involved in cellular processes that can act as druggable targets is a key starting point for drug discovery. However, the most highly expressed cellular targets do not always represent the most biologically relevant targets for therapeutic intervention.

Biological process or phenotypes of mammalian cells, such as those involved in disease are regulated by cellular pathways. The multiplicity of such interactions involved in any given biological process or phenotype, together with the lack of generally applicable tools for their investigation and/or manipulation, presents a formidable challenge to current strategies for discovery of candidate therapeutics

Current screening methods that can identify candidate drug targets linked to disease biology have typically been performed using gene knock-outs. Recently, methods of randomly disrupting gene function by inserting sequences in or near genes have been described (Rad R. et al., Science (2010) 330 (6007):1104-1107).

There remains the need for an efficient method or tools for the identification of druggable targets, particularly small-molecule drug targets.

### BRIEF SUMMARY OF THE DISCLOSURE

In one aspect, the invention provides a method of identifying a genomic target for a test stimulus that is capable of modulating a cell phenotype, comprising:
a) providing a population of cells that are phenotypically negative in response to the test stimulus;
b) modifying the population of cells by random insertion into the cell genome of a gain-in-function construct;
c) contacting the modified cell population with the test stimulus;
d) screening the exposed modified cell population to identify modified cells that are phenotypically positive in response to the test stimulus; and
e) identify a gene or genes associated with said positive phenotype thereby identifying the genomic target of the test stimulus.

Suitably the cell is a mammalian cell.

Suitably the cell comprises a nucleotide sequence encoding for a phenotypically linked selectable marker.

Suitably the phenotype is activation of a cell signalling pathway. Suitably the phenotype is calcium signalling, in certain instances the phenotype is calcium signalling in a nociceptive signalling pathway. Alternatively, the phenotype is nociceptive signalling.

Suitably the construct is an expression cassette. Suitably the expression cassette comprises a promoter. Suitably the expression cassette is a transposon. Suitably the transposon is an activating transposon. Suitably the transposon is a piggyBac transposon.

Suitably the test stimulus is a compound that acts on a nociceptive signalling pathway.

Suitably screening to identify modified cells that are phenotypically positive comprises identification of genotypic differences in modified cells that are phenotypically positive when compared to phenotypically negative control cells.

Suitably identify a gene or genes associated with said positive phenotype comprises next generation sequencing.

Suitably identify a gene or genes associated with said positive phenotype comprises identifying a cell correlating to a barcode.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 provides a schematic outlining an exemplary transposon cassette for us in the invention wherein ITR: Inverted terminal repeats. Pac: Puromycin N-acetyltransferase enzyme. pA: poly-adenylation site UCOE: Universal Chromatin Opening Element.

FIG. 2 provides a schematic of TAGS. Phenotypically negative cells stably expressing a phenotypic selection marker are subjected to transposon-mediated random gene-activating mutagenesis (step 1.), yielding a library of derived clones that newly express random combinations of genes. This clone library is stimulated with the agonist in question, whereby those clones that randomly express the right gene or genes responds, and triggers detectable changes in the phenotypic marker. These positive clones are then selected by cell sorting (Step 2.). Isolated single clonal cells are grown into large, clonal cell populations and genotyped to identify the location of transposon insertion (Step 3.). Common insertion sites (CIS) are then identified, and genes within a 200 kb range of CIS are subjected to secondary confirmation via siRNA and cDNA to unequivocally identify the correct target genes.

FIG. 3 provides genotyping strategy for TAGS. Genomic DNA from clonal populations is isolated and sheared using an ultra-sonication device (Covaris). Custom-made adaptor sequences are ligated onto sheared genomic fragments to enable insertion site-specific amplification. The amplicons from the first PCR reaction are then further amplified using nested primers, which contain a combination of barcodes (see primer list) as well as adaptors for the Illumina NGS platform. The barcoded and adapted amplicons from the second PCR are mixed at equimolar ratios and sent for multiplexed Illumina NGS.

FIG. 4 provides validation of TAGS (AITC-screen for TRPA1). a) a Piggy-Bac library was generated in Hela-C cells, stimulated with 100 uM AITC and co-incident photo-conversion light, and sorted via FACS based on CaMPARI red/green signal. b) Positive clones were collected as single cells, expanded into clonal populations, and testing for AITC responsiveness via Fura-2 calcium imaging. Percent responding cells are shown for each population. c-d) Example Fura-2 imaging traces. c) phenotypic negativity of HeLa-C cells. d) Example traces from population #22 in b. e) Genotyping of 100% responders. Each row shows data from one of 16 example clonal populations. Bottom row shows NCBI-RefSeq. Blue arrows highlight transcription start site (TSS) of TRPA1. Red arrows highlight the true common insertion site (CIS) upstream of the TSS, found in every clone genotyped.

### DETAILED DESCRIPTION

The inventors have provided a novel Transposon Activated Genomewide Screening (TAGS) method, that advantageously provides a new platform for novel gene discovery. The method provides a platform for discovery of novel mechanical, thermal and chemical receptors for a test stimulus.

The inventors have provided a new screening method that can identify candidate drug targets linked to disease biology using a ‘phenotypic’ assay, that screens the impact of a drug or stimulus at a genetic level. Advantageously, the methods allow identification of biologically relevant druggable targets that may not be identifiable using traditional screening approaches.

The methods of the invention involve the use of random gene activation that serves to turn a phenotypically negative cell into a phenotypically positive cell and then uses high throughput sequence analysis to identify the genotypic changes associated with the positive phenotype, thereby identifying novel druggable sites for treatment of disease.

The methods are advantageously unbiased. They involve random positive modulations (e.g. increased expression or activation) of genes in a cell followed by subsequent determination of the impact of said random positive modulations on the cells response to a test drug or stimulus.

The invention thus provides methods of identifying a genomic target for a test stimulus that is capable of modulating a cell phenotype, comprising providing a population of cells that are phenotypically negative in response to the test stimulus; modifying the population of cells by random insertion into the cell genome of a gain-in-function construct; contacting the modified cell population with the test stimulus; screening the exposed modified cell population to identify modified cells that are phenotypically positive in response to the test stimulus; identify a gene or genes associated with said positive phenotype thereby identifying the genomic target of the test stimulus.

Random Forward Mutagenesis

The method of the invention comprises providing a population of cells that are phenotypically negative in response to the test stimulus; and subsequently modifying the population of cells by random insertion into the cell genome of a gain-in-function construct.

Cells useful in the methods are preferably proliferating cell types. The cells may be eukaryotic cells or prokaryotic cells. Suitably, the eukaryotic cells are mammalian cells. Exemplary mammalian cells include HEK 293, Chinese Hamster Ovary cells, HEK 293F, HEK 293H, HEK 293A, HEK 293FT, HEK293T, CHO DG44, CHO-S, CHO-DX611, Expi293F, ExpiCHO-S, T-Rex, Hela, MCF7, COST, NIH 3T3, U2O5, A375, A549, N2A, PGP1 iPS, BHK, Hap1, Jurkat, N0, ARPE19 or MDA-MB-231.

In certain instances, the cells may be derived from a patient or a disease model.

In certain embodiments the cells may be plant cells. The plant cell may be of a crop plant such as cassava, corn, sorghum, wheat, or rice. The plant cell may also be of an algae, tree or vegetable.

Suitably, the cell comprises a nucleotide sequence encoding for a phenotypically linked selectable marker. As used herein, “phenotypically linked selectable marker” means a nucleotide sequence that imparts a distinct phenotype to the host cell expressing the marker upon expression or activation of a particular gene or pathway, in particular a particular gene or pathway linked to a phenotype of interest. Such a nucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the constructs described herein. In certain embodiments the selectable marker is a calcium sensor.

As used herein “phenotype” or “cell phenotype” refers to any detectable characteristic in a cell or population of cell. As used herein a cell or population of cells that is phenotypically positive exhibits a specific detectable characteristic. As used herein a cell or population of cells that is phenotypically negative does not exhibit a specific detectable characteristic. Detectable characteristics include, but are not limited to protein, RNA or DNA in the cell or in sub-compartments of the cell, etc. The phenotype can also be related to cell growth, morphology or cell-cell interactions. In some cases, the phenotype can be temporal changes, dynamics of cellular properties, or the like.

In one embodiment the specific phenotype is selected from the group consisting of cell growth, enzymatic activity, metabolic capacity, resistance chemical or biological agents. In one embodiment, phenotype is activation of a metabolic pathway or a cell signalling pathway.

As used herein, “cell signalling pathway” refers to a series of interacting factors in a cell that transmit an intracellular signal within the cell in response to an extracellular stimulus at the cell surface and leading to changes in cell phenotype. Transmission of signals along a cell signalling pathway may result in the activation of one or more transcription factors which alter gene expression. Preferred cell signalling pathways are pathways known to be involved in disease models such as pain, inflammation, cancer. In one embodiment the phenotype is calcium signalling. In one embodiment the signalling pathway is a nociceptive signalling pathway and suitably, the phenotype is calcium signalling.

A functional cell signalling pathway is a pathway that is intact and capable of transmitting signals, if the pathway is switched on or activated, for example by an appropriate extracellular stimulus. An active cell signalling pathway is a pathway that has been switched on, for example by an appropriate extracellular stimulus and is actively transmitting signals.

The phenotype may be identified or determined using any suitable technique, for example, using optical techniques, through analysis of cell behaviour, or the like. Phenotype may be identified or determined using cell sorting techniques based on viability, fluorescence, radioactivity, magnetic features. For example, the cells may be selected by flow cytometry, or other techniques described herein. In addition, in some embodiments, the phenotype may be determined using a protein or a nucleic acid, for example using a detectably labelled protein nucleic acid. Suitably the phenotype may be determined using Fluorescence Activated Cell Sorting (FACS). Suitably, the phenotype may be determined using image-activated cell sorting (Nitta et al., 2018, Cell 175, 266-276).

Suitably identification of phenotypically positive cells comprises identification of calcium signalling in response to a test stimulus.

As used herein the term “genome” as used herein includes an organism's chromosomal/nuclear genome as well as any mitochondrial, and/or plasmid genome. As used herein, the term “gene” refers to a nucleic acid molecule capable of being used to produce mRNA, antisense RNA, RNAi (miRNA, siRNA, shRNA), anti-microRNA antisense oligodeoxyribonucleotide (AMO), and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions).

As used herein “random insertion” refers to non-biased modification of the genome by insertion at any genomic location, allowing random activation of genes in a given cell or cell population. This allowing for the screening of any desired phenotype and the identification of its associated gene.

As used herein “gain-of-function” construct refers to construct that comprises a nucleic acid sequence that is capable of increasing expression or activity of a gene upon insertion into a genome. In particular, the nucleic acid sequence of the construct is capable of increasing expression or activity of a gene at or near the site of insertion.

As used herein, the term “expression” refers to transcription of a polynucleotide from a DNA template, resulting in, for example, an mRNA or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene product”. Expression may include splicing the mRNA in a eukaryotic cell, if the polynucleotide is derived from genomic DNA.

Suitably the nucleic acid construct is an expression cassette. As used herein the term “expression cassette” is a polynucleotide construct, generated recombinantly or synthetically, comprising regulatory sequences operably linked to a selected polynucleotide to facilitate expression of the selected polynucleotide in a host cell. Regulatory sequences may include activator binding sequences, enhancers, introns, polyadenylation recognition sequences, promoters, transcription start sites, repressor binding sequences, stem-loop structures, translational initiation sequences, internal ribosome entry sites (IRES), translation leader sequences, transcription termination sequences (e.g., polyadenylation signals and poly-U sequences), translation termination sequences, primer binding sites, and the like. As used herein the term “operably linked” refers to polynucleotide sequences or amino acid sequences placed into a functional relationship with one another.

Suitably, the gain-in-function construct is a transposon. As used herein, the term “transposon” a polynucleotide that is able to excise from a donor polynucleotide, for instance, a vector, and integrate itself into an insertion site, for instance, a cell's genomic or extrachromosomal DNA. A transposon includes a polynucleotide that includes a nucleic acid sequence flanked by cis-acting nucleotide sequences; if at least one cis-acting nucleotide sequence is positioned 5′ to the nucleic acid sequence, and at least one cis-acting nucleotide sequence is positioned 3′ to the nucleic acid sequence. Cis-acting nucleotide sequences include at least one ITR at each end of the transposon, to which a transposase binds.

As used herein, the term “transposase” refers to a polypeptide that catalyses the excision of a transposon from a polynucleotide and the subsequent integration of the transposon into the genomic or extrachromosomal DNA of a target cell.

A “vector” is a composition of matter that comprises a nucleic acid of interest. In some embodiments, a vector comprises a transposon and may be used to deliver the transposon to the interior of a cell.

In certain preferred embodiments, the transposon is a mammalian piggyBac transposon. The term “piggyBac transposon” refers to a mobile genetic element that transposes between vectors and chromosomes via a “cut and paste” mechanism. During transposition, the PB transposase recognizes transposon-specific inverted terminal repeat sequences (ITRs) located on both ends of the transposon vector and efficiently moves the contents from the original sites and integrates into TTAA chromosomal sites. The resulting transformed cells or group of cells are stable transformants. In addition to the transposition activity, the ITR may function as an enhancer to stimulate the expression of endogenous genes near the insertion site.

The terms “insertion site” refer to the location of transposition in the DNA. The insertion sites of DNA transposons may be identified by short direct repeats followed by a series of inverted repeats important for the excision by the transposase.

Suitably, the transposon is an activating transposon, which comprises a promoter such that transposon insertion increases the transcription of a gene at or near the insertion site. As used herein the term “promoter” refers to a regulatory element that directs constitutive, inducible, and repressible expression of a polynucleotide sequence in a cell. The promoter may be an inducible promoter, a repressible promoter, and a constitutive promoter.

Transposons can be introduced into a genome (including chromosomal and/or plasmid DNA) by any standard procedures which are well-known to those skilled in the art, such as electroporation (or any other suitable transformation method). For example, non-viral means involving transfection in vitro are of use. Such methods include the use of calcium phosphate, DEAE dextran, electroporation, and protoplast fusion. Liposomes can also be potentially beneficial for delivery of DNA into a cell. Additionally, the non-viral based delivery can be nano-based or aerosolized.

Contacting the Modified Cell Population with the Test Stimulus

The modified cells are subsequently contacted with a test stimulus.

As used herein, “contacting” has its normal meaning and refers to combining two or more molecules (e.g., a test agent and a polypeptide) or combining molecules and cells (e.g., a test agent and a cell). Contacting can occur in vitro, e.g., combining two or more agents or combining a test agent and a cell or a cell lysate in a test tube or other container. Contacting can also occur in a cell or in situ, e.g., contacting two polypeptides in a cell by co-expression in the cell of recombinant polynucleotides encoding the two polypeptides, or in a cell lysate.

As used here in a “test stimulus” refers to an agent or compound and includes polypeptides, nucleic acids, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrrolidines, derivatives, structural analogs or combinations thereof. The test stimulus may be a synthetic compound or a natural compound.

In some embodiments, the test stimulus is a small molecule organic compound, e.g., chemical compounds with a molecular weight of not more than about 1,000 or not more than about 500.

In some embodiments, the test stimulus is a physical stimuli, for example temperature or force.

In some embodiments, the test stimulus is a pathogenic agent, for example a virus or a bacterium.

Selection of Positive Clones and Target Identification

The modified cells contacted with the test stimulus are then screened to identify phenotypically positive cells, using methods well known in the art, such as using high-throughput screening techniques including, but not limited to, marker selection, fluorescence-activated cell sorting (FACS)-based screening platforms, microfluidics-based screening platforms, and the like and combinations thereof.

Identification of a gene or genes associated with a phenotype may be determined using methods know in the art, by for example measuring gene expression or activity. Identification of a gene or genes associated with a phenotype may involve identification of genotypic differences in modified cells that are phenotypically positive when compared to phenotypically negative control cell. The genotypic differences may comprise gene activation, gene insertion, gene knock-down, gene knock-out, or regulatory element deletion. In preferred embodiments that genotypic difference comprises gene activation.

The identification of genotypic differences may comprise measuring or measured differences of DNA, RNA, protein or post translational modification; or measuring or measured differences of protein or post translational modification correlated to RNA and/or DNA level(s); or measuring or detecting expression of a reporter gene or protein.

As used here in the terms “control” and “negative control” refer to unmodified cells that are phenotypically negative. Preferably the unmodified cells are of the same type of cells as the modified phenotypically positive cells.

Identification of a gene or genes associated with a phenotype comprises sequencing. For example methods known to those of skill in the art such as high throughput sequencing methods disclosed in Mitra (1999) Nucleic Acids Res. 27(24):e34; pp. 1-6. Sequencing methods useful in the present disclosure include Shendure et al., Accurate multiplex polony sequencing of an evolved bacterial genome, Science, vol. 309, p. 1728-32. 2005; Drmanac et al., Human genome sequencing using unchained base reads on self-assembling DNA nanoarrays, Science, vol. 327, p. 78-81. 2009; McKernan et al., Sequence and structural variation in a human genome uncovered by short-read, massively parallel ligation sequencing using two-base encoding, Genome Res., vol. 19, p. 1527-41. 2009; Rodrigue et al., Unlocking short read sequencing for metagenomics, PLoS One, vol. 28, e11840. 2010; Rothberg et al., An integrated semiconductor device enabling non-optical genome sequencing, Nature, vol. 475, p. 348-352. 2011; Margulies et al., Genome sequencing in microfabricated high-density picolitre reactors, Nature, vol. 437, p. 376-380. 2005; Rasko et al. Origins of the E. coli strain causing an outbreak of hemolytic-uremic syndrome in Germany, N. Engl. J. Med., Epub. 2011; Huffer et al., Labelled nucleoside triphosphates with reversibly terminating aminoalkoxyl groups, Nucleos. Nucleot. Nucl., vol. 92, p. 879-895. 2010; Seo et al., Four-colour DNA sequencing by synthesis on a chip using photocleavable fluorescent nucleotides, Proc. Natl. Acad. Sci. USA., Vol. 102, P. 5926-5931 (2005); Olejnik et al.; Photocleavable biotin derivatives: a versatile approach for the isolation of biomolecules, Proc. Natl. Acad. Sci. USA., vol. 92, p. 7590-7594. 1995; US 2009/0062129 and US 2009/0191553.

Exemplary next generating sequencing methods known to those of skill in the art include Massively parallel signature sequencing (MPSS), Polony sequencing, pyrosequencing (454), Illumina (Solexa) sequencing by synthesis, SOLiD sequencing by ligation, Ion semiconductor sequencing (Ion Torrent sequencing), DNA nanoball sequencing, chain termination sequencing (Sanger sequencing), Heliscope single molecule sequencing, Single molecule real time (SMRT) sequencing (Pacific Biosciences) and nanopore sequencing.

The identification of a gene or genes associated with a phenotype may include identifying a cell or clone correlating to a barcode.

Suitably, phenotypically positive single cell clones are sorted from the population and expanded prior to analysis to identify the gene or genes associated with the desired phenotype.

In an exemplary embodiment, the methods described herein can be used to identified genes that serve as therapeutic targets for analgesic therapy. In such embodiments, the test stimulus is suitably a compound known to act on a nociceptive signalling pathway. In such embodiments the phenotype is suitably is calcium signalling. In such embodiments, the cell suitably comprises a Ca2+ reporter, for example a fluorescent photoconversion-based calcium sensor, such as CaMPARI2. In such embodiments, the gain-in-function construct is suitably a transposon comprising a promoter, in particular a piggyBac transposon.

### Examples

Aspects of the invention are demonstrated by the following non-limiting examples.

A novel method of Transposon Activated Genomewide Screening (TAGS) is exemplified below.

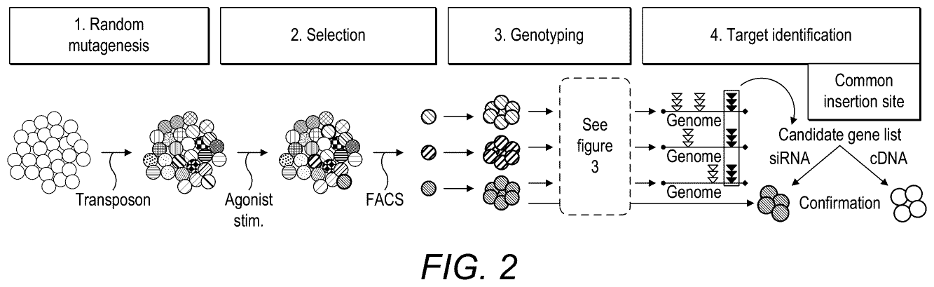

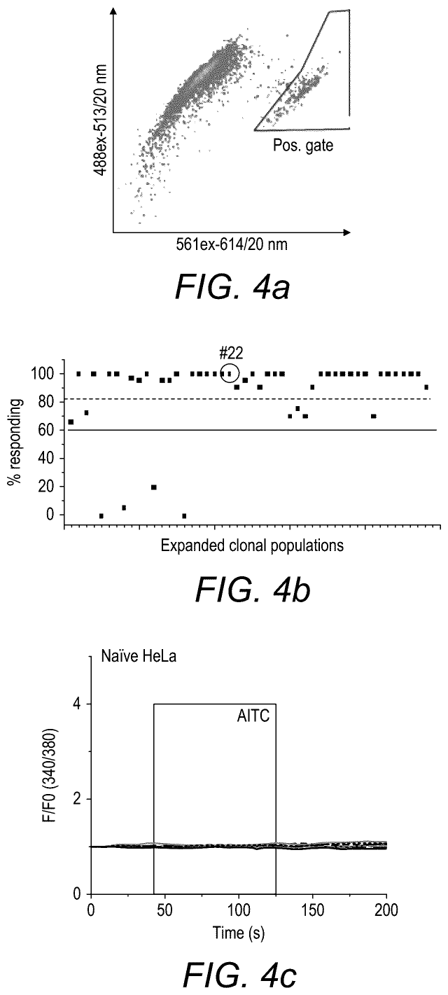

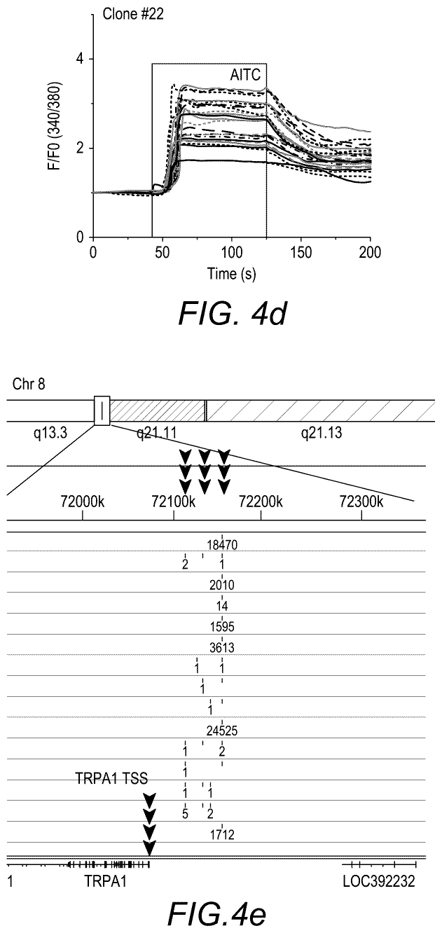

The inventors validated the ability of TAGS to identify genes reliably (FIG. 2). Mustard oil (AITC) is a specific agonist of TRPA1, a calcium-permeable ion channel. TAGS correctly identified TRPA1 as the gene that confers sensitivity to AITC. Newly AITC-responding clones were isolated from a screening library via FACS and AITC responsiveness was independently verified via calcium imaging (FIG. 4 a-d). HeLa cells stably expressing CaMPARI-H396K were used for screening library generation. These are phenotypically negative for AITC responses (FIG. 4d). Genotyping 100%-responders revealed clustered insertions 34-41 kb upstream of the TRPA1 transcription start site (TSS), present in all clones genotyped, implying a single receptor (FIG. 4e). Indeed, TRPA1 knockout mice lose all sensitivity to AITC. As this was the only cluster present in all clones and no other coding genes were in range, secondary confirmation was not necessary in this case.

STEP 1) Random Forward Mutagenesis.

A transposable DNA cassette containing a strong promoter was inserted into the genome of a phenotypically negative cell line, i.e., one that does not functionally express the sought-after gene. This was done with the aid of the PiggyBac (PB) or Tol2 transposase enzymes. Inserted promoters can activate genes as far as 40-60 kilobases. In a library containing ˜10\hat{ }6 unique cellular clones (each with ˜5-20 insertions), the sought-after gene was randomly expressed in a number of clones, which therefore become phenotypically positive.

Generating the Transposon and Transposase Constructs

The inventors used a destabilized PiggyBac (DDPB) or destabilized Tol2 (DDT2) DNA transposase. DDPB was generated by inserting the FKBP-DD domain (synthesized by IDT as a short gene-fragment, listed below as “DD”) into an EcoRI-linearised pCMV-hyPBase plasm id (Wellcome Sanger Institute). DDT2 was generated by PCR-linearization of the pCMV-Tol2 vector (Addgene) with primers DDT2-F/DDT2-R, followed by NEB HiFi assembly with the synthesized DD domain fragment. Stabilization of transposase enzymes was achieved by addition of 300 nM Shield-1 (Generon) to the media.

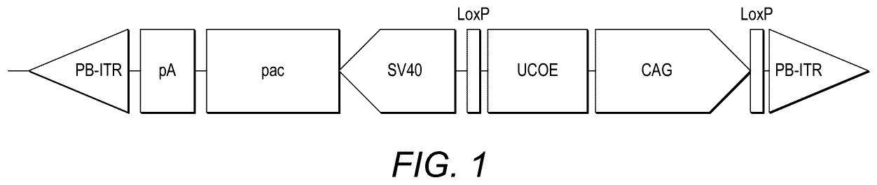

The transposable DNA cassette contains three main elements, flanked by PiggyBac and Tol2 ITR recognition sequences (FIG. 1): 1. the puromycin N-acetyltransferase (Pac) gene driven by an SV40 promoter; 2. The CAG promoter flanked by IoxP sites; 3. a universal Chromosome Opening Element (UCOE), a 1 kb intronic region between the HNRPA2B1-CBX3 housekeeping genes.

The cassette was assembled in a series of 3 cloning steps as follows.

Cloning step 1: 7-fragment NEB HiFi assembly. The vector fragment was generated by PCR from PB-SB-PGKNeo-bpA (Wellcome Sanger) using the V-F/V-R primers. Fragments 1 and 6 (5′ and 3′ ITR recognition sequences for the Tol2 transposase, respectively) were PCR amplified with primers 1-F/1-R and 6-F/6-R from the pKTol2p-PTK plasmid (Addgene). Fragments 2-4 (SV40 promoter, pac gene, SV40 pA signal, respectively) were amplified from the pPUR vector (Clonetech) using primers 2-F/2-R and 3-F/3-R and 4-F/4-R. Fragment 5 (F5) contained two LoxP sites flanking a custom-designed multiple cloning site (MCS) and was synthesized as a small gene fragment by IDT.

Cloning step 2: The CAG promoter insert was subsequently isolated from the pX330 vector (Addgene) using restriction enzymes Agel-Xbal and subcloned into the vector construct above using the same enzyme sites.

Cloning step 3: A 1.5 kb UCOE fragment was introduced upstream of the CAG promoter as follows. Genomic DNA was isolated from 100K HeLa cells using the Qiagen Blood and Tissue kit according to manufacturer's instructions. The UCOE fragment was amplified from the genomic DNA using the primers UCOE-F/UCOE-R and inserted via NEB-HiFi cloning into the vector construct resulting from step 2 linearized with Xbal.

Random Forward Mutagenesis Library Creation:

Transposon constructs were transfected into 10\hat{ }6 cells stably expressing the fluorescent sensor (generation of these described in Step 2) using Lipofectamine 2000 (Thermo) according to manufacturer's instructions in HeLa and A549 cells. An Amaxa Nucleofector I device (Lonza) is used with Cell line Nucleofector kit V (Lonza) for ARPE19 and MDA-MB-231 cells. After a 24 hour incubation period, Shield-1 compound (Generon) was added to the media to stabilize the transposon. 6 Hours later the cells are transfected with the transposon DNA cassette, as before. Shield-1 is maintained in the medium for 24 hours, then removed. After a 3-day incubation period cells are subjected to 1 ug/ml Puromycin (Merck) selection for 2 weeks. Resultant libraries were used immediately for screening (see step 2 below).

PRIMER/CONSTRUCT LIST (in 5′→3′ Orientation):

DD:
(SEQ ID NO: 1)
acggccgccagtgtgctggccaccatgggagtgcaggtggaaaccatctc
cccaggagacgggcgcaccttccccaagcgcggccagacctgtgtggtgc
actacaccgggatgcttgaagatggaaagaaagtcgattcctcccgggac
agaaacaagccctttaagtttatgctaggcaagcaggaggtgatccgagg
ctgggaagaaggggttgcccagatgagtgtgggtcagagagccaaactga
ctatatctccagattatgcctatggtgccactgggcacccaggcatcatc
ccaccacatgccactctcgtcttcgatgtggagcttctaaaaccggaaGC
CGGCGCAatgggcagcagcctgg;
DDT2-F:
(SEQ ID NO: 2)
ggagcttctaaaaccggaagccggcatggaggaagtatgtgattcatc;
DDT2-R:
(SEQ ID NO: 3)
gccagcacactggcggccgttccgcagcttttagagcagaag;
V-F:
(SEQ ID NO: 4)
cacctctgctctctcttcattttattatatattagtcacgatatctataa
caagaaa;
V-R:
(SEQ ID NO: 5)
ctctgggatccagaatgtttaaaagttttgttactttatagaagaaattt
tgagttttt;
1-F:
(SEQ ID NO: 6)
tttaaacattctggatcccagaggtgtaaagtactt;
1-R:
(SEQ ID NO: 7)
cacattccacagtctaatacgactcactatagggagaccaaggg;
2-F:
(SEQ ID NO: 8)
gtcgtattagactgtggaatgtgtgtcagttaggg;
2-R:
(SEQ ID NO: 9)
tggcggcaggcggccgacct;
3-F:
(SEQ ID NO: 10)
ggccgcctgccgccaccatc;
3-R:
(SEQ ID NO: 11)
attatgatcctcgtcaggcaccgggc;
4-F:
(SEQ ID NO: 12)
cggtgcctgacgaggatcataatcagccataccacatttgt;
4-R:
(SEQ ID NO: 13)
atacgaagttataatttcctttttgttaagtgacctaattaacaggag;
F5:
(SEQ ID NO: 14)
ataacttcgtatagcatacattatacgaagttatggcgcgccattctaga
ggtacctaaccggtatccatggagataacttcgtatagcatacattatac
gaagttat;
6-F:
(SEQ ID NO: 15)
tacgaagttatatcaattaaccctcactaaagggagacc;
6-R:
(SEQ ID NO: 16)
cacctctgctctctcttcattttattatatattagtcacgatatctataa
caagaaa;
UCOE-F:
(SEQ ID NO: 17)
aagttatgtaacgggtacctggccctccgcgcctacag;
UCOE-R:
(SEQ ID NO: 18)
cgaagttatggcgcgccattggagacgccgtggccccc

STEP 2) Selection of Positive Clones.

Positive clones in which activation of the target gene has resulted in a phenotypic switch were identified and isolated using Fluorescence Activated Cell Sorting (FACS). This switch was therefore tied to a fluorescent event using specific indicators. For this the inventors used CaMPARI2 fluorescent photoconversion-based calcium sensor. This fluorophore is sensitive to changes in intracellular calcium (a ubiquitous second messenger). CaMPARI2 converts permanently converting from green to red fluorescence if calcium elevation and 405 nm light stimulation coincide. Mutagenized cell libraries were therefore stimulated with the agonist in question, while simultaneously exposed to 120 mW/cm\hat{ }2 405 nm photoconversion light from a custom-made UV-LED light source.

Generation of Screening Cell Lines Stably Expressing the Fluorescent Reporter CaMPARI

The inventors purchased the adenoviral AAV-CaMPARI2 construct (Addgene) and subcloned it into the mammalian pT3-Neo plasmid, along with a CMV promoter to drive its expression using a 3-fragment NEB HiFi cloning as follows. The vector fragment was amplified from pT3-Neo using primer pair pT3-F/pT3-R. Fragment 1 containing a CMF promoter was amplified from a pCMV vector (Clonetech) with primer par F1-F/F1-R. Fragment 2 was amplified from the AAV-CamPARI2 construct using primers F2-F/F2-R.

The inventors used site-directed mutagenesis (Q5 NEB, according to kit manufacturer's instructions) to generate three derived constructs, each containing point mutation(s) in CaMPARI2 that decrease its calcium-sensitivity. Thus, the inventors crated CaMPARI2-H396K, CaMPARI2-F391W-G395D, and CaMPARI2-L398T. Each of these constructs was stably inserted into the genome of four cell lines: HeLa, A549, ARPE19, and MDA-MB-231 cells (original lines purchased from ATCC). These stable lines were created by co-transfecting our constructs with the pCMV(CAT)T7-SB100 vector (Addgene) to induce rapid and robust transgenesis. Cells stably incorporating the fluorescent CaMPARI construct were selected via FACS sorting using a Melody cell sorter (BD) 3 weeks after transfection. 1 mg/ml G418 (Merck) selection pressure was applied 5 days past transfection to limit growth of un-transfected cells. The resulting 12 stable cell lines are stored cryo-preserved. For each screening experiment, these lines are tested for phenotypic negativity, and the most suitable line is used for TAGS for the specific stimulus in question.

PRIMERS (in 5′→3′ Orientation)

pT3-F:
(SEQ ID NO: 19)
aacaacaattgctaattaagatctcgagggaatgaaagacccc;
pT3-R:
(SEQ ID NO: 20)
aatcaatgtcaacaactagtatcgatatgcatgctttgca;
F1-F:
(SEQ ID NO: 21)
tcgatactagttgttgacattgattattgactagttattaatagtaatca
attacggg;
F1-R:
(SEQ ID NO: 22)
agcttgaattcgaagctctgcttatatagacctcccac;
F2-F:
(SEQ ID NO: 23)
ataagcagagcttcgaattcaagctgctagcaaggatcc;
F2-R:
(SEQ ID NO: 24)
gagatcttaattagcaattgttgttgttaacttgtttattgcag

Creation of Custom-Made UV-LED Light Source.

A 100 W UV-LED chip (Chanzon) was attached to a Parabolic reflector (StratusLED) and an Aluminium Heat Sink with Cooling Fan (Tesfish). A 3000 mA Constant-Current LED driver (Chanzon) is used to power the LED. The circuit is controlled by an FRM01 Timer Relay for precise photoconversion timing. The parabolic mirror is placed upside down on a reflective surface, yielding uniform light intensity inside this compartment of ˜120 mW/cm\hat{ }2 at the level of the cells.

Selection of Cells During Screening

The mutagenized library from STEP 1 was subjected to the stimulus co-incident with photo-conversion using our custom UV-LED light source for 35 seconds. Cells were then washed, trypsinzied, resuspended in FACS buffer (2% Fetal Bovine Serum, 2 mM EDTA in Dulbecco's Phosphate Buffered Saline) containing 3 ug/ml DAPI (Merck). Positive cells were selected using a Melody FACS sorter (BD) based on Red/Green fluorescence intensities.

3) Genotyping.

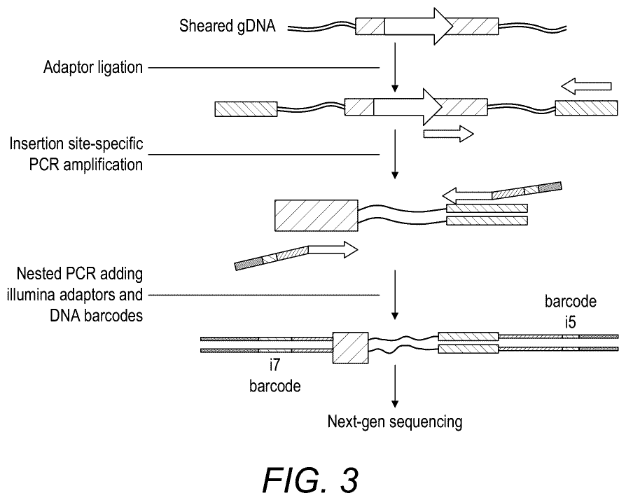

FACS-isolated single clones were grown into clonal populations. Positive responses to the stimuli were confirmed in each cell line, followed by validation of the dependence of this new positive phenotype on the promoter inserted via the use of Cre-recombinase. Genomic DNA was then isolated from each confirmed population. Insertion-site specific PCR was conducted to amplify transposon-adjacent genomic regions. These were tagged with DNA barcodes in a second PCR step and genotyped in parallel using the Illumina MiSEQ Next-Gen Sequencing platform (FIG. 3)

Validation of a Positive Phenotype Dependent Upon the Inserted Promoter

A test sample from each positive clonal line was loaded with the fluorescent calcium indicator Fura-2AM for 30 minutes in regular media supplemented with 1 ul/ml PluronicF127, washed with Ringer solution, and tested for positivity for the phenotype in question using conventional fluorescent microscopy (see FIG. 4a-d). A further test sample from confirmed positive clonal lines was transfected (as described above) with the pCAG-Cre vector (Addgene) to induce transient Cre recombinase expression. Phenotypic positivity was then re-tested as above, and populations in which the phenotype persists are discarded. Clonal populations in which Cre-mediated excision of the CAG promoter reverses the phenotypic switch are genotyped as follows.

Library Preparation and Genotyping

Genomic DNA was isolated from each confirmed clonal population using a 96-well gDNA isolation kit (Zymo) according to manufacturer's instructions. Genomic DNA was sheared in a Covaris machine to a target size of ˜300 basepairs according to manufacturer's instructions. Sheared genomic DNA is purified using Ampure XP magnetic beads (BD) according to manufacturer's instructions at a 1:1 ratio (size optimised for ˜400 bp left and right selection). Splinkerette adaptors (Duplexed from primers SPLNK-LE and SPLNK-HP) were annealed to fragmented DNA using the NEBNext Ultra II ligation and end repair module (NEB). Ligated fragments were purified with Ampure XP beads. Insertion-specific PCR is performed on purified fragments using the 5-1-F/SPL-1-R primer pair. Products were purified with Ampure XP beads. A second PCR step was performed with arrayed primer pairs **5-2-P50*/SPL-2-P70*. See primer list below. Products are purified with Amure XP beads and multiplexed for MiSEQ (performed in a core facility according to manufacturer's instructions).

PRIMERS (in 5′→3′ Orientation. Asterisk Denotes a Phosphorothioesther Bond)

SPLNK-HP:
(SEQ ID NO: 25)
5′GGAATTCTCGGGTGCCAAGGAACTCCAGTCACTTTTTTTTTTCAAAAAAAAAA
SPLNK-LE:
(SEQ ID NO: 26)
5′GTTCCCATGGTACTACTCATAGTGACTGGAGTTCCTTGGCACCCGAGAATTCCA*T
5-1-F:
(SEQ ID NO: 27)
5′ CGCTATTTAGAAAGAGAGAGCAATATTTC*A
SPL-1-R:
(SEQ ID NO: 28)
5′ GTTCCCATGGTACTACTCAT*A
PB5-2-P501:
(SEQ ID NO: 29)
5′AATGATACGGCGACCACCGAGATCTACATATAGCCTACACTCTTTCCCTACACGACGC
TCTTCCGATCTCATGCGTCAATTTTACGCAGACTAT*C
PB5-2-P502:
(SEQ ID NO: 30)
5′AATGATACGGCGACCACCGAGATCTACAATAGAGGCACACTCTTTCCCTACACGACG
CTCTTCCGATCTCATGCGTCAATTTTACGCAGACTAT*C
PB5-2-P503:
(SEQ ID NO: 31)
5′AATGATACGGCGACCACCGAGATCTACACCTATCCTACACTCTTTCCCTACACGACGC
TCTTCCGATCTCATGCGTCAATTTTACGCAGACTAT*C
PB5-2-P504:
(SEQ ID NO: 32)
5′AATGATACGGCGACCACCGAGATCTACAGGCTCTGAACACTCTTTCCCTACACGACG
CTCTTCCGATCTCATGCGTCAATTTTACGCAGACTAT*C
PB5-2-P505:
(SEQ ID NO: 33)
5′AATGATACGGCGACCACCGAGATCTACAAGGCGAAGACACTCTTTCCCTACACGACG
CTCTTCCGATCTCATGCGTCAATTTTACGCAGACTAT*C
PB5-2-P506:
(SEQ ID NO: 34)
5′AATGATACGGCGACCACCGAGATCTACATAATCTTAACACTCTTTCCCTACACGACGC
TCTTCCGATCTCATGCGTCAATTTTACGCAGACTAT*C
PB5-2-P507:
(SEQ ID NO: 35)
5′AATGATACGGCGACCACCGAGATCTACACAGGACGTACACTCTTTCCCTACACGACG
CTCTTCCGATCTCATGCGTCAATTTTACGCAGACTAT*C
PB5-2-P508:
(SEQ ID NO: 36)
5′AATGATACGGCGACCACCGAGATCTACAGTACTGACACACTCTTTCCCTACACGACGC
TCTTCCGATCTCATGCGTCAATTTTACGCAGACTAT*C
T25-2-P501:
(SEQ ID NO: 37)
5′AATGATACGGCGACCACCGAGATCTACATATAGCCTACACTCTTTCCCTACACGACGC
TCTTCCGATCTatttttgagtactttttacacctct*g
T25-2-P502:
(SEQ ID NO: 38)
5′AATGATACGGCGACCACCGAGATCTACAATAGAGGCACACTCTTTCCCTACACGACG
CTCTTCCGATCTatttttgagtactttttacacctct*g
T25-2-P503:
(SEQ ID NO: 39)
5′AATGATACGGCGACCACCGAGATCTACACCTATCCTACACTCTTTCCCTACACGACGC
TCTTCCGATCTatttttgagtactttttacacctct*g
T25-2-P504:
(SEQ ID NO: 40)
5′AATGATACGGCGACCACCGAGATCTACAGGCTCTGAACACTCTTTCCCTACACGACG
CTCTTCCGATCTatttttgagtactttttacacctct*g
T25-2-P505:
(SEQ ID NO: 41)
5′AATGATACGGCGACCACCGAGATCTACAAGGCGAAGACACTCTTTCCCTACACGACG
CTCTTCCGATCTatttttgagtactttttacacctct*g
T25-2-P506:
(SEQ ID NO: 42)
5′AATGATACGGCGACCACCGAGATCTACATAATCTTAACACTCTTTCCCTACACGACGC
TCTTCCGATCTatttttgagtactttttacacctct*g
T25-2-P507:
(SEQ ID NO: 43)
5′AATGATACGGCGACCACCGAGATCTACACAGGACGTACACTCTTTCCCTACACGACG
CTCTTCCGATCTatttttgagtactttttacacctct*g
T25-2-P508:
(SEQ ID NO: 44)
5′AATGATACGGCGACCACCGAGATCTACAGTACTGACACACTCTTTCCCTACACGACGC
TCTTCCGATCTatttttgagtactttttacacctct*g
SPL-2-P701:
(SEQ ID NO: 45)
5′CAAGCAGAAGACGGCATACGAGATCGAGTAATGTGACTGGAGTTCCTTGG*C
SPL-2-P702:
(SEQ ID NO: 46)
5′CAAGCAGAAGACGGCATACGAGATTCTCCGGAGTGACTGGAGTTCCTTGG*C
SPL-2-P703:
(SEQ ID NO: 47)
5′CAAGCAGAAGACGGCATACGAGATAATGAGCGGTGACTGGAGTTCCTTGG*C
SPL-2-P704:
(SEQ ID NO: 48)
5′CAAGCAGAAGACGGCATACGAGATGGAATCTCGTGACTGGAGTTCCTTGG*C
SPL-2-P705:
(SEQ ID NO: 49)
5′CAAGCAGAAGACGGCATACGAGATTTCTGAATGTGACTGGAGTTCCTTGG*C
SPL-2-P706:
(SEQ ID NO: 50)
5′CAAGCAGAAGACGGCATACGAGATACGAATTCGTGACTGGAGTTCCTTGG*C
SPL-2-P707:
(SEQ ID NO: 51)
5′CAAGCAGAAGACGGCATACGAGATAGCTTCAGGTGACTGGAGTTCCTTGG*C
SPL-2-P708:
(SEQ ID NO: 52)
5′CAAGCAGAAGACGGCATACGAGATGCGCATTAGTGACTGGAGTTCCTTGG*C
SPL-2-P709:
(SEQ ID NO: 53)
5′CAAGCAGAAGACGGCATACGAGATCATAGCCGGTGACTGGAGTTCCTTGG*C
SPL-2-P710:
(SEQ ID NO: 54)
5′CAAGCAGAAGACGGCATACGAGATTTCGCGGAGTGACTGGAGTTCCTTGG*C
SPL-2-P711:
(SEQ ID NO: 55)
5′CAAGCAGAAGACGGCATACGAGATGCGCGAGAGTGACTGGAGTTCCTTGG*C
SPL-2-P712:
(SEQ ID NO: 56)
5′CAAGCAGAAGACGGCATACGAGATCTATCGCTGTGACTGGAGTTCCTTGG*C

4) Target identification. Insertions that are causative of the phenotypic switch occur in clusters, i.e. close to insertion sites found in all other selected clones in case of a single receptor, or in ˜50% of the clones in case of two receptors, etc. Such positive clusters were identified using a combination of bioinformatics methods, comparing the probability of insertions to a negative dataset (randomly selected negative clones from the original library). Genes near clusters of significant enrichments as compared to the negative control populate a shortlist for secondary verification to unequivocally identify the true causative gene(s). Secondary verification is done by a combination of siRNA knockdown in the clonal populations (confirms necessity of a gene(s) for the phenotype) as well as cDNA-mediated expression in the parental, naïve cell line (confirms sufficiency of the gene(s) to confer the phenotype).

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

What is claimed is:

1. A method of identifying a genomic target for a test stimulus that is capable of modulating a cell phenotype, comprising:
a) providing a population of cells that are phenotypically negative in response to the test stimulus;
b) modifying the population of cells by random insertion into the cell genome of a gain-in-function construct;
c) contacting the modified cell population with the test stimulus;
d) screening the exposed modified cell population to identify modified cells that are phenotypically positive in response to the test stimulus; and
e) identify a gene or genes associated with said positive phenotype thereby identifying the genomic target of the test stimulus.

2. The method according to claim 1 wherein the cell is a mammalian cell.

3. The method according to claim 1, wherein the cell comprises a nucleotide sequence encoding for a phenotypically linked selectable marker.

4. The method according to claim 1, wherein the phenotype is activation of a cell signalling pathway.

5. The method according to claim 4, wherein the cell signalling pathway is a nociceptive signalling pathway.

6. The method according to claim 4, wherein the phenotype is calcium signalling.

7. The method according to claim 1, wherein the construct is an expression cassette.

8. The method according to claim 7, wherein the expression cassette comprises a promoter.

9. The method according to claim 7, wherein the expression cassette is a transposon.

10. The method according to claim 9, wherein the transposon is an activating transposon.

11. The method according to claim 9, wherein the transposon is a piggyBac transposon.

12. The method according to claim 1, wherein the test stimulus is a compound that acts on a nociceptive signalling pathway.

13. The method according to claim 1, wherein screening to identify modified cells that are phenotypically positive comprises identification of genotypic differences in modified cells that are phenotypically positive when compared to phenotypically negative control cells.

14. The method according to claim 1, wherein identify a gene or genes associated with said positive phenotype comprises next generation sequencing.

15. The method according to claim 1, wherein identify a gene or genes associated with said positive phenotype comprises identifying a cell correlating to a barcode.

16. A method of identifying a genomic target for a test stimulus that is capable of modulating a cell phenotype as described herein with reference to the accompanying drawings.
